(19) United States
(12) Patent Application Publication
(10) Pub. No.: US 2023/0041196 A1
(43) Pub. Date: Feb. 9, 2023

(54) WEARABLE CHRONIC MONITORING SYSTEMS, METHODS, AND DEVICES

(71) Applicant: ARIZONA BOARD OF REGENTS ON BEHALF OF THE UNIVERSITY OF ARIZONA, Tucson, AZ (US)

(72) Inventor: PHILIPP GUTRUF, Tucson, AZ (US)

(73) Assignee: Arizona Board of Regents on Behalf of the University of Arizona, Tucson, AZ (US)

(21) Appl. No.: 17/791,523

(22) PCT Filed: Jan. 11, 2021
(86) PCT No.: PCT/US2021/012918

§ 371 Date: Jul. 7, 2022

Related U.S. Application Data
(60) Provisional application No. 62/959,883, filed on Jan. 11, 2020.

(51) Int. Cl. A61B 5/00; A41D 13/12; A61B 5/0205; H02J 50/20

(52) U.S. Cl. CPC A61B 5/6831; A41D 13/1281; A61B 5/0205; A61B 5/0002; H02J 50/20; A61B 2562/164; A61B 2562/066; A61B 2560/0242; H02J 2310/23

(57) ABSTRACT

The present disclosure is directed to systems and methods of collecting environmental and/or biometric information and/or data using a chronic monitoring apparatus that includes a wearable expandable support structure to wirelessly receive power via a wireless power transfer antenna disposed in, on, or about the wearable expandable support structure. The chronic monitoring apparatus includes power receiver circuitry, data transmission circuitry, sensor circuitry, and control circuitry. The wearable expandable support structure maintains close contact between at least a portion of the sensor circuitry and the wearer of the chronic monitoring apparatus without requiring the use of adhesives or other bonding agents. The chronic monitoring apparatus communicates the collected environmental and/or biometric information to external data collection circuitry. The components included in the chronic monitoring apparatus are sealed within the wearable expandable support structure providing a rugged, reliable, resilient and waterproof system that is biocompatible, non-irritating and does not require the use of adhesives.

### TECHNICAL FIELD

The present disclosure relates to wearable monitors, more specifically to wearable biometric monitoring devices.

### BACKGROUND

Chronic monitoring is performed in a variety of commercial, industrial, and medical settings. Typically, such monitoring includes placing one or more sensors (often an array that includes a number of sensors) on or about a portion of an individual's body or limbs. The sensors collect biometric and/or environmental data that is communicated on a periodic or continuous basis to monitoring and/or recording equipment. The chronic monitoring equipment typically includes batteries or a similar power source to power the sensors and to transmit data to the monitoring equipment. Current chronic monitoring systems are typically battery powered devices that are affixed to the wearer's skin using an adhesive that renders the monitoring apparatus sensitive to motion artifacts, particularly where electrical contact between the monitoring system and the wearer's skin is needed. Furthermore, such battery powered devices are often burdensome and uncomfortable to wear for extended periods of time, leading to individuals prematurely removing the monitoring apparatus because of the need to recharge or because of discomfort.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 is a schematic of an illustrative system that includes a chronic monitoring apparatus, a wireless power transmitter, and data collection circuitry, in accordance with at least one embodiment described herein;

FIG. 2 is a plane view of an illustrative chronic monitoring apparatus disposed about the forearm of a user, in accordance with at least one embodiment described herein;

FIG. 3 is a schematic diagram of an illustrative system in which the data collection circuitry forwards the environmental and/or biometric data collected by the chronic monitoring apparatus to a remote system via a network, in accordance with at least one embodiment described herein;

FIG. 4 is a flow diagram of an illustrative method of fabricating a chronic monitoring apparatus, in accordance with at least one embodiment described herein; and

FIG. 5 is a high level flow diagram of an illustrative method of operating a chronic monitoring apparatus, in accordance with at least one embodiment described herein.

Although the following Detailed Description will proceed with reference being made to illustrative embodiments, many alternatives, modifications and variations thereof will be apparent to those skilled in the art.

### DETAILED DESCRIPTION

The systems and methods described herein provide a chronic monitoring apparatus and a chronic monitoring system that includes a chronic monitoring apparatus that receives energy via far field power harvesting to beneficially minimize or even eliminate the need for an energy storage device within the chronic monitoring apparatus. The chronic monitoring apparatus may include a wearable, expandable, support structure that places a sensor array in close proximity to a user's body or limbs and/or in proximity to an ambient environment about the user without requiring the use of potentially allergenic adhesives or tape to maintain the chronic monitoring apparatus in a desired location on the user. The wearable expandable support structure may include an elastomeric material having mechanical properties similar to the epidermis. The chronic monitoring apparatus includes a three-dimensional far field harvesting antenna formed in, on, or about the wearable, expandable, mesh structure, beneficially eliminating the need for cumbersome external antennas. The chronic monitoring apparatus may, in some instances, be capable of transdermal delivery of one or more therapeutic substances, light therapy, thermal actuation or haptic feedback. In such instances, the chronic monitoring apparatus may monitor one or more biometric parameters that are communicated to one or more network connected remote devices (e.g., a server or cloud-based device).

The chronic monitoring apparatus may include any number and/or combination of biometric and/or environmental sensors. Example biometric sensors include: pulse oximetry sensors, heart rate sensors, skin/dermal conductivity sensors, electromyograph sensors; or electrocardiograph sensors. Example environmental sensors include: an accelerometer, a temperature sensor, a gyroscopic sensor, a barometric pressure sensor, a humidity sensor, volatile organic compound (VOC) sensor, particle sensor, oxygen sensor, carbon monoxide sensor, or a compound/class of compound sensor. Beneficially, a variety of sensors may be coupled to the wearable, expandable, mesh structure in locations that provide highest sensing fidelity and such that interference between the sensors is reduced or eliminated. The chronic monitoring apparatus includes an RF transmitter, such as a Bluetooth Low Energy or Near Field Communication transmitter, to communicate the data generated by the sensor array to a nearby receiver, such as a monitoring system or smartphone. The chronic monitoring apparatus may be used in a variety of medical, commercial, industrial, and athletic applications, such as medical monitoring, physical performance monitoring, environmental exposure monitoring, or combinations thereof.

A chronic monitoring apparatus is provided. The chronic monitoring apparatus may include a wearable expandable support structure having an inner surface and an outer surface; a wireless power harvesting antenna disposed in at least a portion of the expandable matrix structure; power receiver circuitry physically coupled to the support matrix structure and conductively coupled to the wireless power transfer antenna; a sensor circuitry physically coupled to the support matrix structure; and control circuitry communicatively coupled to the plurality of sensors and physically coupled to the support matrix structure.

A method of fabricating a wearable chronic monitoring apparatus is provided. The method may include: disposing a wireless power transfer antenna in a wearable expandable support structure having an inner surface and an outer surface; conductively coupling the wireless power transfer antenna to power receiver circuitry coupled to the wearable expandable support structure; conductively coupling sensor circuitry to the power conversion circuitry; conductively coupling control circuitry to the power conversion circuitry; and conductively coupling the control circuitry and the power conversion circuitry to RF transmitter circuitry.

A chronic monitoring system is provided. The system may include: wireless RF power generation circuitry; wireless RF receiver circuitry; and a wearable chronic monitoring apparatus, that includes: a wearable expandable support structure having an inner surface and an outer surface; a wireless power transfer antenna disposed in at least a portion of the wearable expandable support structure; power receiver circuitry physically coupled to the wearable expandable support structure and conductively coupled to the wireless power transfer antenna; sensor circuitry physically coupled to the support matrix structure; and control circuitry communicatively coupled to the sensor circuitry and physically coupled to the wearable expandable support structure.

A chronic monitoring method is provided. The method may include: receiving, by a wireless power transfer antenna disposed in a wearable expandable support structure, an RF signal from an wireless power transmitter; converting, by power receiver circuitry disposed in the wearable expandable support structure, the received RF signal to electrical power; distributing the electrical power to sensor circuitry and to data transmission circuitry; receiving, by the data transmission circuitry disposed in the wearable expandable support structure, one or more signals that include at least one of environmental or biometric information from the sensor circuitry; and communicating, by the data transmission circuitry, one or more signals that include the received environmental or biometric information to an external data receiver.

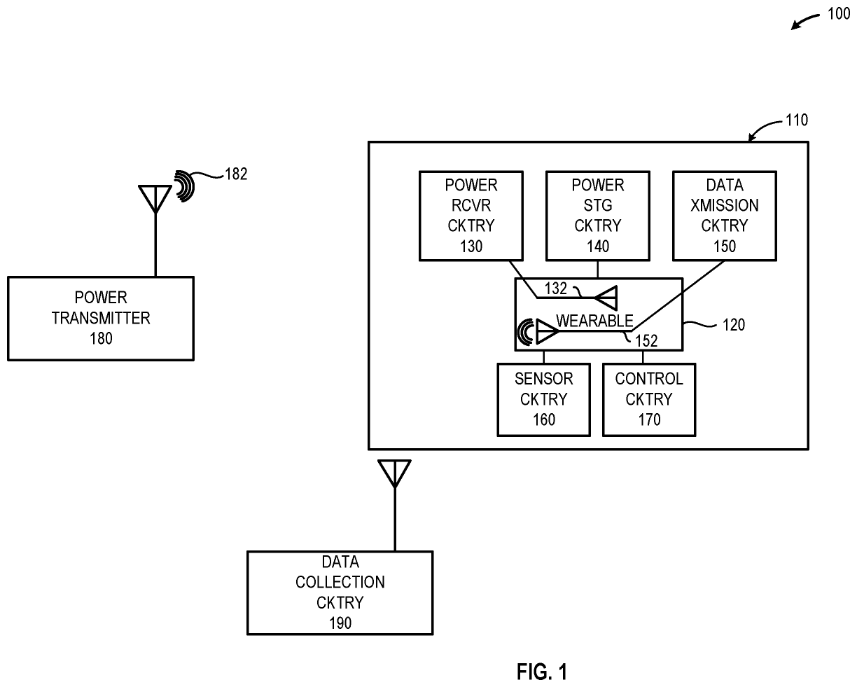

FIG. 1 is a schematic of an illustrative system 100 that includes a chronic monitoring apparatus 110, a wireless power transmitter 180, and data collection circuitry 190, in accordance with at least one embodiment described herein. As depicted in FIG. 1, the chronic monitoring apparatus 110 may include a wearable expandable support structure 120. The chronic monitoring apparatus 110 may include one or more of: wireless power receiver circuitry 130, power storage circuitry 140, data transmission circuitry 150, sensor circuitry 160, and/or control circuitry 170. In embodiments, one or more wireless power antennas 132 to receive wireless power transmissions from the wireless power transmitter 180 may be disposed in, on, or about the wearable expandable support structure 120. In embodiments, the one or more wireless power antennas 132 may include a three dimensional (3D) structure all or a portion of which may include or otherwise incorporate features, such as coils or pleats, that allow the one or more wireless power antennas 132 to expand with the expandable support structure 120. In embodiments, one or more data transmission antennas 152 to transmit information and/or data collected by the sensor circuitry 160 to one or more external data collection circuits 190 may be disposed in, on, or about the wearable expandable support structure 120.

In operation, the power transmitter 180 transmits an RF signal 182 that is received by the power receiver circuitry 130. The power receiver circuitry 130 converts at least a portion of the received RF signal 182 to an electrical current. In some embodiments, all or a portion of the electrical current produced by the power receiver circuitry 130 may be used by one or more components, such as the data transmission circuitry 150, sensor circuitry 160, and/or control circuitry 170 coupled to the wearable expandable support structure 120. In some embodiments, all or a portion of the electrical current produced by the power receiver circuitry 130 may be stored in the power storage circuitry 140. In at least some embodiments, the power receiver circuitry 130 may include some or all of the power storage circuitry 140.

Each of the sensors included in the sensor circuitry 160 generates information and/or data on a continuous, periodic, aperiodic, or event-driven basis. In some embodiments, the information and/or data generated by the sensor circuitry 160 may be communicated to the data collection circuitry 190 on a real-time or near real-time basis. In some embodiments, the information and/or data generated by the sensor circuitry 160 may be communicated to the data collection circuitry 190 in the form of a periodic, aperiodic, or continuous data stream or in the form periodic, aperiodic, or continuous data bursts. In some embodiments, the sensor circuitry 160 and/or the control circuitry 170 may include data storage circuitry to store or otherwise retain all or a portion of the information and/or data generated by the sensor circuitry 160.

The data transmission circuitry 150 may include one or more of: encryption/decryption circuitry, data compression circuitry, error detection/correction circuitry, or combinations thereof. The data transmission circuitry 150 may communicate information and/or data to the data collection circuitry 190 using one or more currently available and/or future developed standard communication protocols. For example, BLUETOOTH® Low Energy (BLE) or Near Field Communication (NFC) communication protocols.

The wearable expandable support structure 120 provides the structural backbone of the chronic monitoring apparatus 110. The wearable expandable support structure 120 may be fabricated to conform to any portion of a user's body or limbs. For example, the wearable expandable support structure 120 may be fabricated to conform to all or a portion of a user's arm, leg, hand, foot, or torso. The wearable expandable support structure 120 may be fabricated using any biocompatible, expandable, flexible, and/or elastomeric material. In at least some embodiments, the wearable expandable support structure 120 may be fabricated using thermoplastic polyurethane (TPU) or similar elastomeric material. The wearable expandable support structure 120 may be produced using any currently available or future developed molding or fabrication technology such as three-dimensional (3D) printing, injection molding, and similar.

In embodiments, the wearable expandable support structure 120 may be fabricated in the form of a uniform or non-uniform mesh type material or fabric that includes “islands” at some or all of the intersection points. Some or all of the to support some or all of the “islands” may be used to house or accommodate the various sensors and components. In some embodiments, the islands may include generally circular structures having a diameter of 5 millimeters (mm) or less; 7 mm or less; 10 mm or less; or 15 mm or less. In embodiments, the wireless power antenna 132 may be disposed in, on, or about all or a portion of the wearable expandable support structure 120 to provide a 3D wireless power antenna 132. Beneficially, the wearable expandable support structure 120 provides a platform that maintains the sensor array in close proximity to the skin of the user.

The power receiver circuitry 130 includes any number and/or combination of currently available and/or future developed electronic components, optical components, semiconductor devices, and/or logic elements capable of converting RF energy received via the wireless power antenna 132 to an electrical current useful for powering the data transmission circuitry 150, the sensor circuitry 160, and/or the control circuitry 170. In embodiments, the power receiver circuitry 130 is coupled to one or more wireless power antennas 132 having a physical configuration capable of wirelessly receiving power radiated by the power transmission circuitry 180. In embodiments, the power receiver circuitry 130 and the one or more wireless power antennas 132 may wirelessly receive energy radiated by the power transmission circuitry 180 up to a distance of about: 3 meters or less; 6 meters or less; 10 meters or less; 15 meters or less; 25 meters or less; or 50 meters or less. In some embodiments, the power receiver circuitry 130 may include circuitry, systems, and/or devices capable of receiving power over a wireless local area network (WLAN), such as power over WiFi. In such instances, the one or more wireless power antennas 132 may include one or more WiFi (IEEE 802.11) compliant antennas.

In embodiments, the one or more wireless antennas 132 include a three dimensional (3D) structure all or a portion of which may include or otherwise incorporate features, such as coils or pleats, that allow the one or more wireless power antennas 132 to expand with the expandable support structure 120. In embodiments, the power receiver circuitry 130 may include circuitry, systems, and/or components capable of wirelessly receiving energy via one or more microwave signals having a frequency of from about 300 MHz (A=1 meter) to 300 GHz (A=1 millimeter). For example, in at least some embodiments, the power receiver circuitry 130 may include circuitry capable of receiving a microwave signal in the 915 MHz ISM band. In embodiments, the power receiver circuitry 130 may include circuitry, systems, and/or components capable of wirelessly receiving energy via power over WiFi

In embodiments, all or a portion of the power receiver circuitry 130 may be at least partially encapsulated in the wearable expandable support structure 120. In embodiments, all or a portion of the power receiver circuitry 130 may be completely encapsulated in an island physically coupled to the wearable expandable support structure 120. In embodiments, the power receiver circuitry 130 may include one or more photovoltaic devices (e.g., micro solar cells) or similar to replace or supplement the RF wireless power received by the power receiver circuitry 130.

The power storage circuitry 140 includes any number and/or combination of currently available and/or future developed electronic components, optical components, semiconductor devices, and/or logic elements capable of storing electrical energy. In embodiments, the power receiver circuitry 130 may include all or a portion of the power storage circuitry 140. In embodiments, the power storage circuitry 140 may include any type of electrical storage device including, but not limited to one or more secondary cells, one or more supercapacitors, one or more ultracapacitors, or combinations thereof.

The data transmission circuitry 150 includes any number and/or combination of currently available and/or future developed electronic components, optical components, semiconductor devices, and/or logic elements capable of generating an RF signal that includes information and/or data provided by some of all of the sensor circuitry 160. In embodiments, the data transmission circuitry 150 may include BLUETOOTH® Low Energy (BLE) circuitry, Near Field Communication (NFC) circuitry, or any combination thereof. In embodiments, the data transmission circuitry 150 may be conductively coupled to one or more data transmission antennas 132. In embodiments, the one or more data transmission antennas may be disposed in, on, or about the wearable expandable support structure 120.

In embodiments, the data transmission circuitry 150 may communicate information and/or data to the data collection circuitry 190 on a continuous, intermittent, periodic, or aperiodic basis. In embodiments, the data transmission circuitry 150 may communicate information and/or data to the data collection circuitry 190 on an event-driven basis. For example, the data transmission circuitry 150 may communicate information and/or data to the data collection circuitry 190 when the chronic monitoring apparatus 110 is brought within a defined communication range of the data collection circuitry 190. In embodiments, all or a portion of the data transmission circuitry 150 may be at least partially encapsulated in the wearable expandable support structure 120. In embodiments, all or a portion of the data transmission circuitry 150 may be completely encapsulated in an island physically coupled to the wearable expandable support structure 120.

The sensor circuitry 160 includes any number and/or combination of currently available and/or future developed electronic components, optical components, semiconductor devices, and/or logic elements capable of generating one or more output signals that contain, carry, convey, transport, or otherwise include information and/or data representative of one or more biometric and/or environmental parameters. In embodiments, the sensor circuitry 160 may include any number and or combination of sensors, signal conditioners, signal converters, and/or signal amplifiers. In embodiments, the sensor circuitry 160 may include one or more biometric sensors, one or more environmental sensors, or any combination thereof. In embodiments, the one or more biometric sensors included in the sensor circuitry 160 may be disposed on an interior or inner surface of the wearable expandable support structure 120 such that the respective biometric sensor is disposed proximate a wearer of the wearable expandable support structure 120. In embodiments, the one or more biometric sensors included in the sensor circuitry 160 may be disposed on an outside or external surface of the wearable expandable support structure 120 such that the respective biometric sensor is disposed distal from or spaced apart from the wearer of the wearable expandable support structure 120.

The sensor circuitry 160 may include one or more biometric sensors. Such biometric sensors may include but are not limited to: one or more pulse oximetry sensors; one or more heart rate sensors; one or more electromyograph sensors; and/or one or more electrocardiograph sensors. The sensor circuitry 160 may include one or more environmental sensors. Such environmental sensors may include but are not limited to: one or more accelerometers, one or more temperature sensors, one or more gyroscopic sensors, one or more barometric pressure sensors, one or more humidity sensors, one or more chemical detection sensors, one or more radiation detection sensors, or one or more thermal conductivity sensors. In embodiments, the sensors 160 may be positioned on the wearable expandable support structure 120 in locations that minimize or even eliminate interference between the sensors.

The control circuitry 170 includes any number and/or combination of currently available and/or future developed electronic components, optical components, semiconductor devices, and/or logic elements capable of causing the sensor circuitry 160 to collect biometric and/or environmental information and/or data and causing the data transmission circuitry 150 to communicate the collected biometric and/or environmental information and/or data to the data collection circuitry 190. In embodiments, the control circuitry 170 may cause the storage of the collected biometric and/or environmental information and/or data in one or more data storage devices or circuitry. In embodiments the control circuitry 170 may include one or more Application Specific Integrated Circuits (ASICs); one or more Reduced Instruction Set Computers (RISCs); one or more Field Programmable Gate Arrays (FPGAs); or combinations thereof. In embodiments, the control circuitry 170 may include some or all of the data transmission circuitry 150 and/or data storage circuitry.

The power transmission circuitry 180 includes any number and/or combination of currently available and/or future developed electronic components, optical components, semiconductor devices, and/or logic elements capable of wirelessly communicating an RF signal to the chronic monitoring apparatus 110. In embodiments, the power transmission circuitry 180 may include one or more beamsteering power transmission circuits. In embodiments, the power transmission circuitry 180 may generate a 915 MHz RF ISM signal to provide wireless power to the chronic monitoring apparatus 110. In embodiments, the power transmission circuitry 180 may wirelessly transmit 1 Watt (W) or more; 3 W or more; 5 W or more; or 10 W or more to the power receiver circuitry 130 disposed in the chronic monitoring apparatus 110. In embodiments, the power transmission circuitry 180 may include circuitry, systems, and/or components capable of generating and wirelessly transmitting energy using one or more microwave signals having a frequency of from about 300 MHz (λ=1 meter) to 300 GHz (λ=1 millimeter). For example, the power receiver circuitry 130 may receive energy via one or more microwave signals having a frequency of: about 300 MHz to about 200 GHz; about 300 MHz to about 100 GHz; about 300 MHz to about 10 GHz; about 300 MHz to about 1 GHz; about 500 MHz to about 1 GHz; or about 750 MHz to about 1 GHz. In embodiments, the power transmission circuitry 180 may include circuitry, systems, and/or components capable of transmitting and receiving wireless local area network, such as IEEE 802.11 compliant (WiFi) communications while contemporaneously generating and wirelessly transmitting energy via the local area network. In

The data collection circuitry 190 includes any number and/or combination of currently available and/or future developed electronic components, optical components, semiconductor devices, and/or logic elements capable of receiving the signal containing the sensor information and/or data generated by the data transmission circuitry 150 in the chronic monitoring apparatus 110. In embodiments, the data collection circuitry 190 may include a BLUETOOTH Low Energy (BLE) or Near Field Communication (NFC) receiver. In embodiments, the data collection circuitry 190 may be disposed in a processor-based device such as a laptop computer, a handheld computer, a wearable computer, a smartphone, or a desktop computer. In embodiments, the data collection circuitry 190 may receive information and/or data from the data transmission circuitry 150 on a continuous, intermittent, periodic or aperiodic basis. In embodiments, the data collection circuitry 190 may receive information and/or data from the data transmission circuitry 150 on an event-driven basis—for example, when the chronic monitoring apparatus 110 is carried within a defined range of the data collection circuitry 190. In embodiments, the data collection circuitry 190 may pull information and/or data from the data transmission circuitry 150. In other embodiments, the data collection circuitry 190 may push information and/or data to the data transmission circuitry 150. In embodiments, all or a portion of the data collection circuitry 190 may be disposed within the same housing or enclosure as the power transmission circuitry 180.

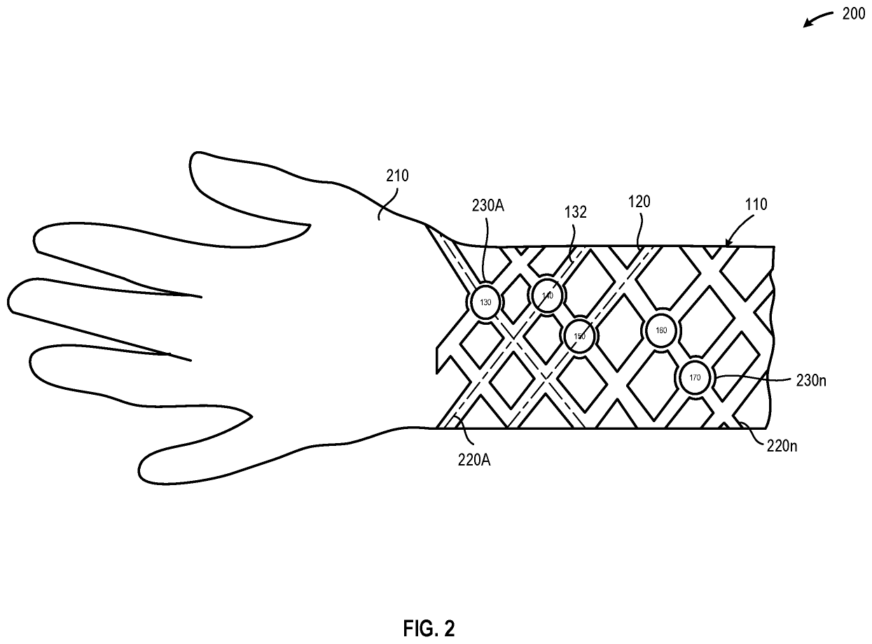

FIG. 2 is a plane view of an illustrative chronic monitoring apparatus 110 disposed about the forearm of a user 210, in accordance with at least one embodiment described herein. Although depicted in FIG. 2 as disposed about a forearm of a system user 210, the chronic monitoring apparatus 110 may be disposed about any portion of a limb such as an arm or leg, an extremity such as a foot or hand, or about all or a portion of a torso of the system user 210. As depicted in FIG. 2, the chronic monitoring apparatus 110 may include a mesh type wearable expandable support structure 120 that includes a plurality of intersecting, flexible, expandable members 220A-220n (collectively, “expandable members 220”). One or more islands 230A-230n (collectively, “islands 230”) may be formed at the intersection of two or more expandable members 220.

As depicted in FIG. 2, each of one or more of the wireless power receiver circuitry 130, the power storage circuitry 140, the data transmission circuitry 150, the sensor circuitry 160, and/or the control circuitry 170 may be disposed on one or more islands 230 formed in the wearable expandable support structure 120. In embodiments, each island 230 may partially or completely encapsulate some or all of the circuitry disposed thereupon. Beneficially, each island 230 may be disposed about all or a portion of any circuitry such that the circuitry is water-resistant or water-proof, thereby reducing or even eliminating the need for the wearer to remove the chronic monitoring apparatus 110 during activities involving immersion or wetting (e.g., swimming, bathing, showering). In embodiments, each of the islands 230 may have the same or a different diameter. In embodiments, each of the islands may have a diameter of about: 1 millimeter (mm) or less; 3 mm or less; 5 mm or less; 7 mm or less; 10 mm or less; or 15 mm or less. In embodiments, the islands 230 may be formed integral with the expandable members 230. For example, the islands 230 may be 3D printed with the expandable members 220 to provide the wearable expandable support structure 120. As depicted in FIG. 2, in embodiments, some or all of the wireless power antenna 132 may be partially or completely embedded or disposed in, on, or, about the expandable members 220 and/or islands 230 forming the wearable expandable support structure 120.

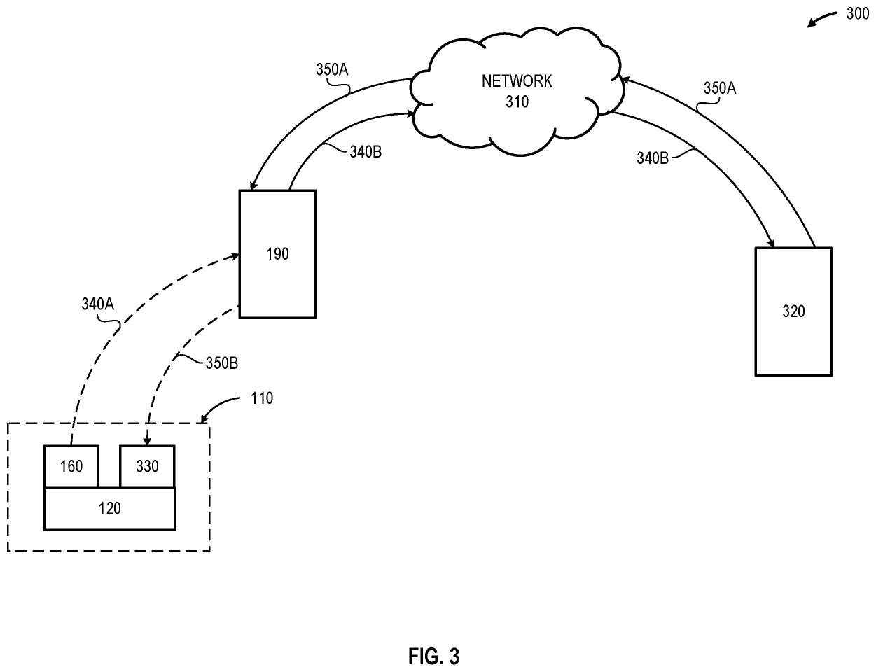

FIG. 3 is a schematic diagram of an illustrative system 300 in which the data collection circuitry 190 forwards the environmental and/or biometric data collected by the chronic monitoring apparatus 110 to a remote system 320 via a network 310, in accordance with at least one embodiment described herein. As depicted in FIG. 3, the chronic monitoring apparatus 110 may include one or more output circuits 330, such as a transdermal delivery circuitry. In such embodiments, the remote system 320 may analyze otherwise use the received environmental and/or biometric data to determine one or more actions to be undertaken by the output circuitry 330.

In embodiments, the wearable expandable support structure 120 may include output circuitry 330 to perform one or more actions on or to the wearer 210 of the chronic monitoring apparatus 110. For example, the wearable expandable support structure 120 may include one or more output circuitry 330 having one or more transdermal delivery systems. In embodiments, the frequency and/or quantity of feedback and/or material delivery to the wearer of the chronic monitoring apparatus 110 may be based, in whole or in part, on the environmental and/or biometric data collected by the chronic monitoring apparatus 110 and to the remote system 320. The data collection circuitry 190 may communicatively couple to one or more remote devices 320 via one or more networks 310. In embodiments, the one or more networks 310 may include one or more local area networks (LANs); one or more wireless local area networks (WLANs); one or more metropolitan area networks (MANs); one or more wide area networks (WANs); one or more wireless wide area networks (WWANs); one or more worldwide networks (e.g., the Internet); or any combination thereof.

The one or more remote devices 320 may include one or more servers or similar processor-based devices. In embodiments, the one or more remote devices 320 may include one or more cloud based servers. The one or more remote devices 320 may include circuitry, for example artificial intelligence (AI) circuitry capable of determining a treatment regimen using the environmental and/or biometric data collected by the chronic monitoring apparatus 110 and communicated to the one or more remote devices 320 via the data collection circuitry 190. For example, in some embodiments, the one or more remote devices 320 may include biomedical diagnostic circuitry capable of determining a therapeutic regimen based upon biometric data collected by the chronic monitoring apparatus 110.

For example, one or more sensors included in the sensor circuitry 160 may communicate 340A information and/or data indicative or representative of one or more biometric parameters associated with a wearer 210 of the chronic monitoring apparatus 110 to the data collection circuitry 190. The data collection circuitry 190 may communicate 340B, via network 310, the information and/or data indicative or representative of one or more biometric parameters associated with a wearer 210 to remote biomedical analysis circuitry disposed in a cloud based server 320. The chronic monitoring apparatus 110 may include “low-power” devise (e.g., low power microcontrollers, etc.) to extend battery recharge rates, etc. The biomedical analysis circuitry communicates 350A to the data collection circuitry 190 one or more outputs/actions to be performed by output circuitry 330 included in the chronic monitoring apparatus 110. The data collection circuitry 190 then communicates 350B the one or more actions to be performed by the output circuitry 330 to the chronic monitoring apparatus control circuitry 170. The chronic monitoring apparatus control circuitry 170 then causes the output circuitry 330 to perform the desired action.

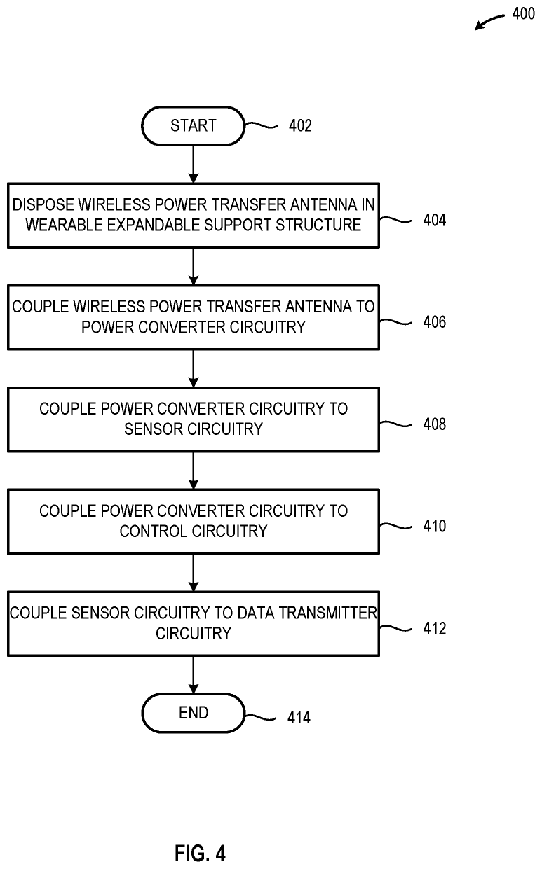

FIG. 4 is a flow diagram of an illustrative method 400 of fabricating a chronic monitoring apparatus 110, in accordance with at least one embodiment described herein. The method 400 commences at 402.

At 404, the wireless power transfer antenna 132 is disposed in, on, or about all or a portion of the wearable expandable support structure 120. In embodiments, all or a portion of the wireless power transfer antenna 132 may be encapsulated within all or a portion of the wearable expandable support structure 120. In other embodiments, all or a portion of the wireless power transfer antenna 132 may be disposed in, on, or about the surface of the wearable expandable support structure 120. Advantageously, disposing the wireless power transfer antenna 132 within or proximate the wearable expandable support structure 120 provides a three-dimensional antenna to receive the wireless power transfer from the power transmission circuitry 180. In embodiments, the one or more wireless power transfer antennas 132 may be grounded to the skin surface of a user wearing the expandable support structure 120 via the power receiver circuitry 130.

At 406, the wireless power transfer antenna 132 is coupled to the power receiver circuitry 130. In embodiments, the power receiver circuitry 130 may be physically coupled to the wearable expandable support structure 120. For example, the power receiver circuitry 130 may be disposed in, on, or about an island or similar structure that is physically coupled to or formed with the wearable expandable support structure 120.

At 408, the sensor circuitry 160 couples to the power receiver circuitry 130. The sensor circuitry 160 receives power from the power receiver circuitry 130 and may include any number of environmental and/or biometric sensing devices. In embodiments, the sensor circuitry 160 may receive all or a portion of the operating power from one or more energy storage devices 140, such as a secondary battery or ultracapacitor coupled to the power receiver circuitry 130.

At 410, the control circuitry 170 couples to the power receiver circuitry 130. In embodiments, all or a portion of the control circuitry 170 may be incorporated into the sensor circuitry 160. In embodiments, the control circuitry 170 may include data storage circuitry to receive and store environmental and/or biometric data from the sensor circuitry 160. In some embodiments, the data storage circuitry may include one or more volatile or nonvolatile and or removable storage devices (one or more micro-SD cards, flash memory, etc.)

At 412, the sensor circuitry 160 couples to the data transmission circuitry 150 to enable the communication of collected environmental and/or biometric data to the data collection circuitry 190. In embodiments, the sensor circuitry 160 may couple directly to the data transmission circuitry 150 such that the data transmission circuitry receives all or a portion of the information and/or data generated by the sensor circuitry 160 directly from the sensor circuitry 160. In other embodiments, the sensor circuitry 160 may indirectly couple to the data transmission circuitry 150. In such embodiments, the information and/or data generated by the sensor circuitry 160 may be filtered, conditioned, amplified, stored, otherwise pass through one or more intermediate components disposed between the sensor circuitry 160 and the data transmission circuitry 150. The method 400 concludes at 414.

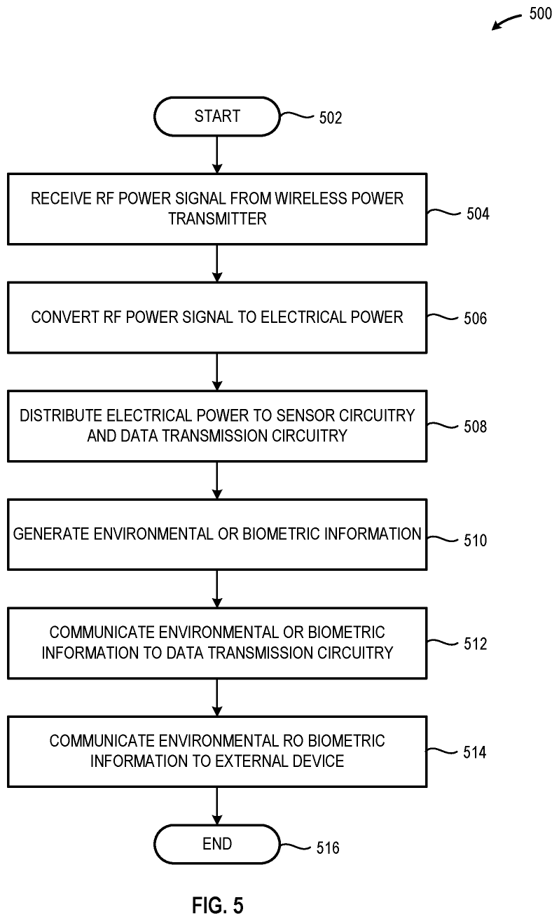

FIG. 5 is a high level flow diagram of an illustrative method 500 of operating a chronic monitoring apparatus 110, in accordance with at least one embodiment described herein. In embodiments, the chronic monitoring apparatus 110 may be used to monitor one or more environmental parameters or conditions in the ambient environment proximate the wearer of the chronic monitoring apparatus 110. In embodiments, the chronic monitoring apparatus 110 may be used to monitor one or more biometric parameters associated with the wearer of the cam 110. The method 500 commences at 502.

At 504, the power receiver circuitry 130 receives an RF signal 182 from power transmission circuitry 180 disposed remote from the power receiver circuitry 130. In embodiments, the RF signal 182 may include a 915 MHz ISM wireless power signal. In embodiments, the power receiver circuitry 130 receives the RF signal 182 via a wireless power transfer antenna 132 that is disposed in, on, or about the wearable expandable support structure 120 to which the power receiver circuitry 130 is physically coupled. In embodiments, the power receiver circuitry 130 may receive about 3 Watts of power via the RF signal 182.

At 506, the power receiver circuitry 130 converts at least a portion of the energy received via the RF signal 182 to electrical power. In embodiments, the power receiver circuitry 130 may include one or more energy storage devices 140, such as one or more secondary cells, one or more ultracapacitors, one or more supercapacitors, or combinations thereof. In such embodiments, at least a portion of the electrical power provided by the power receiver circuitry 130 may be stored or otherwise retained by the one or more energy storage devices 140.

At 508, the power receiver circuitry 130 distributes electrical power to one or more of: the data transmission circuitry 150, the sensor circuitry 160, and/or the control circuitry 170. In embodiments, excess power provided by the power receiver circuitry 130 may be stored or otherwise retained in one or more energy storage devices 140.

At 510, the sensor circuitry 160 generates one or more signals that include information and/or data indicative or representative of one or more environmental parameters and/or one or more biometric parameters. In embodiments, the sensor circuitry 160 may include one or more environmental sensors/sensor arrays, one or more biometric sensors/sensor arrays, or any combination thereof. In embodiments, all or a portion of the sensor circuitry 160 (e.g., one or more biometric sensors) may be disposed in, on, or about the wearable expandable support structure 120 in locations proximate the skin surface of a wearer of the chronic monitoring apparatus 110. In embodiments, all or a portion of the sensor circuitry 160 (e.g., one or more environmental sensors) may be disposed in, on, or about the wearable expandable support structure 120 in locations distal from the skin surface of the wearer of the chronic monitoring apparatus 110. In embodiments, the sensor circuitry 160 may include one or more signal conditioners, signal converters, filters, amplifiers, signal repeaters, or any combination thereof.

At 512, the collected biometric and/or environmental information and/or data is provided to the data transmission circuitry 150 for communication to the data collection circuitry 190. In embodiments, the sensor circuitry 160 may communicate all or a portion of the collected biometric and/or environmental information and/or data directly to the data transmission circuitry 150. In embodiments, one or more data storage devices may communicate all or a portion of the stored collected biometric and/or environmental information and/or data directly to the data transmission circuitry 150.

At 514, the data transmission circuitry 150 communicates the biometric and/or environmental information and/or data to the data collection circuitry 190. In embodiments, the data transmission circuitry 150 may communicate the biometric and/or environmental information and/or data to the data collection circuitry 190 on a continuous basis. In embodiments, the data transmission circuitry 150 may communicate the biometric and/or environmental information and/or data to the data collection circuitry 190 on an intermittent basis. In embodiments, the data transmission circuitry 150 may communicate the biometric and/or environmental information and/or data to the data collection circuitry 190 on a periodic basis. In embodiments, the data transmission circuitry 150 may communicate the biometric and/or environmental information and/or data to the data collection circuitry 190 on an aperiodic basis. In embodiments, the data transmission circuitry 150 may communicate the biometric and/or environmental information and/or data to the data collection circuitry 190 on an event-driven basis. Such events may include but are not limited to: an occurrence of a change in biological parameters (e.g., heart rate, ECG, EKG) of the wearer of the chronic monitoring apparatus 110. Such events may include but are not limited to: an occurrence of an environmental event such as movement of the chronic monitoring apparatus 110 to within a designated range of the data collection circuitry 190.

In embodiments, the biometric and/or environmental information and/or data may be communicated using one or more standard communication protocols, such as a BLUETOOTH® Low Energy (BLE) protocol; a Zigbee communication protocol; a LoRa communication protocol; Near Field Communication (NFC) communication protocol; or a Sigfox communication protocol. The method 500 concludes at 516.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

As used in any embodiment herein, the terms “system” or “module” may refer to, for example, software, firmware and/or circuitry configured to perform any of the aforementioned operations. Software may be embodied as a software package, code, instructions, instruction sets and/or data recorded on non-transitory computer readable storage mediums. Firmware may be embodied as code, instructions or instruction sets and/or data that are hard-coded (e.g., nonvolatile) in memory devices.

As used in any embodiment herein, the term “circuitry” may comprise, for example, singly or in any combination, hardwired circuitry, programmable circuitry such as computer processors comprising one or more individual instruction processing cores, state machine circuitry, and/or firmware that stores instructions executed by programmable circuitry or future computing paradigms including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more mediums (e.g., non-transitory storage mediums) having stored therein, individually or in combination, instructions that when executed by one or more processors perform the methods. Here, the processor may include, for example, a server CPU, a mobile device CPU, and/or other programmable circuitry. Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), embedded multimedia cards (eMMCs), secure digital input/output (SDIO) cards, magnetic or optical cards, or any type of media suitable for storing electronic instructions. Other embodiments may be implemented as software executed by a programmable control device.

Thus, the present disclosure is directed to systems and methods of collecting environmental and/or biometric information and/or data using a chronic monitoring apparatus that includes a wearable expandable support structure to wirelessly receive power via a wireless power transfer antenna disposed in, on, or about the wearable expandable support structure. The chronic monitoring apparatus includes power receiver circuitry, data transmission circuitry, sensor circuitry, and control circuitry. The wearable expandable support structure maintains close contact between at least a portion of the sensor circuitry and the wearer of the chronic monitoring apparatus without requiring the use of adhesives or other bonding agents. The components included in the chronic monitoring apparatus are sealed within the wearable expandable support structure providing a rugged, reliable, resilient and waterproof system.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed:

1. A chronic monitoring apparatus, comprising:
a support structure having an inner surface and an outer surface, wherein the support structure includes a unitary, three-dimensional (3D), wearable, expandable, member;
an expandable 3D wireless power transfer antenna coupled to at least a portion of the support structure;
power receiver circuitry physically coupled to the support structure and conductively coupled to the wireless power transfer antenna;
sensor circuitry physically coupled to the support structure, the sensor circuitry disposed in one or more locations within the support structure such that at least a portion of the sensor circuitry falls proximate a skin surface of a user after the support structure is donned by the user; and
control circuitry communicatively coupled to the plurality of sensors and physically coupled to the support structure.

2. The apparatus of claim 1 wherein the control circuitry further comprises data storage circuitry to store data received from the sensor circuitry.

3. The apparatus of claim 1 wherein the control circuitry further comprises data transmission circuitry to communicate data received from at least some of the sensor circuitry to one or more data collection circuits.

4. The apparatus of claim 3 wherein the data transmission circuitry includes at least one of: Bluetooth Low Energy (BLE) compliant circuitry; Zigbee compliant circuitry; LoRa compliant circuitry; Near Field Communication (NFC) compliant circuitry; or Sigfox compliant circuitry.

5. The apparatus of claim 1 wherein the sensor circuitry includes one or more biometric sensors disposed on the wearable expandable support structure such that at least a portion of the one or more biometric sensors is exposed on the inner surface of the wearable expandable support structure.

6. The apparatus of claim 1 wherein the wearable expandable support structure comprises a support structure that includes that at least one of: a thermoplastic polyurethane (TPU), or silicone.

7. The apparatus of claim 1 wherein the power receiver circuitry further comprises at least one energy storage device.

8. The apparatus of claim 7 wherein the at least one energy storage device comprises a supercapacitor.

9. The apparatus of claim 1 wherein the wireless power transfer antenna comprises a three-dimensional (3D) antenna to wirelessly receive power in the 915 MHz band.

10. The apparatus of claim 1 wherein the power receiver circuitry further comprises one or more photovoltaic devices disposed on at least a portion of the exterior surface of the wearable expandable support structure.

11. The apparatus of claim 1 wherein the sensor circuitry comprises one or more biometric sensors.

12. The apparatus of claim 11 wherein the one or more biometric sensors include at least one of: a pulse oximetry sensor; a heart rate sensor; an electromyograph sensor; or an electrocardiograph sensor.

13. The apparatus of claim 1 the sensor circuitry comprises one or more environmental sensors.

14. The apparatus of claim 13 wherein the one or more environmental sensors include at least one of: an accelerometer, a temperature sensor, a gyroscopic sensor, a barometric pressure sensor, a humidity sensor, a compound/class of compound sensor, or a thermal conductivity sensor.

15. A method of fabricating a wearable chronic monitoring apparatus, the method comprising:
coupling a three-dimensional (3D), expandable, wireless power transfer antenna to at least a portion of a support structure, wherein the support structure includes a unitary, three-dimensional (3D), wearable, expandable, member;
conductively coupling the 3D expandable, wireless power transfer antenna to power receiver circuitry coupled to the support structure;
conductively coupling sensor circuitry to the power conversion circuitry, the sensor circuitry disposed in one or more locations within the support structure such that at least a portion of the sensor circuitry falls proximate a skin surface of a user after the support structure is donned by the user;
conductively coupling control circuitry to the power conversion circuitry; and
conductively coupling the control circuitry and the power conversion circuitry to RF transmitter circuitry.

16. The method of claim 15 wherein conductively coupling the wireless power transfer antenna to the power receiver circuitry further comprises:
conductively coupling the wireless power transfer antenna to power receiver circuitry that includes one or more energy storage devices.

17. The method of claim 15 wherein conductively coupling the control circuitry to the power conversion circuitry further comprises:
conductively coupling control circuitry that includes data storage circuitry to the power conversion circuitry.

18. The method of claim 15 wherein conductively coupling the sensor circuitry to the power conversion circuitry further comprises:
conductively coupling one or more of a plurality of biometric sensors to the power conversion circuitry.

19. A chronic recording system, comprising:
wireless RF power generation circuitry;
wireless RF receiver circuitry; and
a wearable chronic monitoring apparatus, that includes:
a support structure having an inner surface and an outer surface, wherein the support structure includes a unitary, three-dimensional (3D), wearable, expandable, member;
an expandable 3D wireless power transfer antenna coupled to at least a portion of the support structure;
power receiver circuitry physically coupled to the support structure and conductively coupled to the wireless power transfer antenna;
sensor circuitry physically coupled to the support structure, the sensor circuitry disposed in one or more locations within the support structure such that at least a portion of the sensor circuitry falls proximate a skin surface of a user after the support structure is donned by the user; and
control circuitry communicatively coupled to the plurality of sensors and physically coupled to the support structure.

20. A chronic monitoring method, comprising:
receiving an RF signal from an wireless power transmitter at an expandable, three-dimensional (3D) wireless power transfer antenna coupled to a support structure, wherein the support structure includes a unitary, three-dimensional (3D), wearable, expandable, member;
converting, by power receiver circuitry disposed in the support structure, the received RF signal to electrical power;
distributing the electrical power to data transmission circuitry and to sensor circuitry disposed in one or more locations within the support structure such that at least a portion of the sensor circuitry falls proximate a skin surface of a user after the support structure is donned by the user;
receiving, by the data transmission circuitry disposed in the wearable expandable support structure, one or more signals that include at least one of environmental or biometric information from the sensor circuitry; and
communicating, by the data transmission circuitry, one or more signals that include the received environmental or biometric information to an external data receiver.
